(19) United States
(12) Patent Application Publication
(10) Pub. No.: US 2009/0088415 A1
(43) Pub. Date: Apr. 2, 2009

(54) Controlled release pharmaceutical compositions

(75) Inventors: Rajesh Jain, New Delhi (IN); Kour Chand Jindal, New Delhi (IN); Sukhjeet Singh, New Delhi (IN)

Correspondence Address: LADAS & PARRY LLP, 26 WEST 61ST STREET, NEW YORK, NY, 10023, US

(73) Assignee: PANACEA BIOTEC LTD.

(21) Appl. No.: 11/482,185

(22) Filed: Jul. 6, 2006

Related U.S. Application Data
(63) Continuation-in-part of application No. PCT/IN05/00004, filed on Jan. 5, 2005.

(30) Foreign Application Priority Data
Jan. 6, 2004 (IN) 23/DEL/2004
Jan. 6, 2004 (IN) 28/DEL/2004

(51) Int. Cl. A61K 31/545; A61K 31/43; A61K 31/455

(52) U.S. Cl. 514/192; 514/200; 514/197; 514/356

(57) ABSTRACT

A non-disintegrating, non-eroding, non-bioadhesive and non-swelling oral controlled release pharmaceutical composition and process for preparation of such compositions is provided which comprises at least one high dose water soluble active ingredient, at least one diluent, at least one binder, and a polymer system comprising of at least one release controlling polymer wherein the composition formulated into a suitable dosage form maintains its geometric shape even after the drug has diffused from the dosage form and provides the concentrations of active ingredient above effective levels for extended periods of time, optionally with other pharmaceutically acceptable excipients. The compositions preferably comprise antibiotic(s) as active ingredient, more preferably Amoxicillin or its pharmaceutically acceptable salts, hydrates, polymorphs, esters, and derivatives thereof, most preferably amoxicillin sodium, either alone or in combination with other antibiotic(s). Also described are controlled release compositions which provide an initial burst release of approximately 20%-40% of the active ingredient within one hour for achieving blood levels equivalent to minimum inhibitory concentration, while maintaining these levels for an extended period of time.

### FIELD OF THE INVENTION

The present invention relates to controlled release pharmaceutical compositions comprising at least one high dose water soluble active ingredient, and process for preparation of such compositions, preferably comprising antibiotic(s) as active ingredient, more preferably Amoxicillin sodium either alone or in combination with other antibiotic(s). The controlled release compositions are of non-disintegrating, non-eroding, non-bioadhesive and non-swelling type, intended to retain its geometrical shape throughout its transit in the gastro-intestinal tract.

The controlled release composition is useful in providing therapeutically effective levels of the said active ingredient for extended periods of time. Moreover the said composition is expected not to compromise the bioavailability of the active ingredient under fed or fasted conditions.

### BACKGROUND OF THE INVENTION

Amoxicillin is a beta-lactam widely used as a broad-spectrum antibiotic for treatment of a variety of common bacterial infections. Amoxicillin has known susceptibility to inhibition by beta-lactamases produced by resistant organisms. Amoxicillin is available in a variety of formulations, for instance as capsules, tablets, dry powders for reconstitution, chewable tablets, dispersible tablets etc. Amoxicillin is available as tablets of different strengths such as 250 mg, 500 mg, 875 mg, etc. The standard adult dose is 250 mg to 500 mg three times a day (tid). In addition, the 875 mg tablet is intended for dosing twice daily (bid) instead of 500 mg tid. A high dose of 3 g, bid is recommended for treatment of recurrent purulent infection of respiratory tract. Use of 1 g Amoxicillin is recommended as one arm of combination therapy, for eradication of helicobacter pylori in peptic ulcer disease.

In the past, attempts have been made to develop modified release/controlled release formulations of Amoxicillin. Such modified/controlled release tablets may provide better patient compliance since they need to be administered twice daily as compared to the 500 mg dose given tid.

European patent number EP1044680 discloses bilayered tablets comprising of an immediate release dose of a part of Amoxicillin and Potassium clavulanate and a controlled release dose of a second part of Amoxicillin. The controlled release layer is a hydrophilic matrix. The above said composition suffers from the drawback that it requires excess quantities of excipients for preparing bilayered tablets. This combined with the high dose of Amoxicillin results in a product which is too bulky and difficult to administer.

U.S. Pat. No. 5,690,959 discloses a composition prepared using hydrophobic material manufactured by a process of thermal infusion. Amoxicillin, being temperature sensitive, may undergo degradation if subjected to high temperatures for longer periods of time.

U.S. Pat. No. 6,399,086 discloses a pharmaceutical composition of Amoxicillin wherein 50% of the drug is released within 3-4 hours. The said composition is based on hydrophilic erodible polymers.

U.S. Pat. No. 6,368,635 discloses a solid matrix composition which is solid at ambient temperature, which comprises a viscogenic agent, such as an acrylic acid polymer, capable of developing viscosity on contact with water, as dispersed at least in the neighborhood of the surface layer of a matrix particle containing a polyglycerol fatty acid ester or a lipid and an active ingredient. The matrix may be such that a matrix particle containing a polyglycerol fatty acid ester or a lipid and an active ingredient has been coated with a coating composition containing at least one viscogenic agent. Such composition can adhere to the digestive tract and remain there for a prolonged period of time, thereby increasing the bioavailability of the active ingredient. Such gastric mucosa-adherent particles have unpredictable residence time in the stomach and are highly influenced by the gastric contents. Bioavailabilities of active agents from such compositions are highly variable.

European Pat. No. EP0526862 discloses a pharmaceutical composition of Amoxicillin with prolonged residence due to high density of the composition. The said composition suffers from the drawback that non-uniform release of active ingredient results due to variable passage of tablet into intestine by virtue of density itself resulting in significant bioavailability loss.

The PCT Publication No. WO 200384510 describes specifically bilayered tablet formulation comprising antihistaminic decongestant combination. The second discrete zone of the bilayered tablet comprises a decongestant drug and a second carrier base material, the second carrier base material comprising, a mixture of at least one sustained release compound and at least one pharmaceutically accepted glidants or lubricants, wherein the second carrier base material provides the sustained release of decongestant. The said publication does not necessitate the use of at least one diluent and a binder along with a polymer system comprising of at least one release controlling polymer to obtain a non-disintegrating, non-eroding, non-bioadhesive and non-swelling oral controlled release pharmaceutical composition, wherein the drug releases preferably by diffusion.

The PCT Publication No. WO 2004012700 relates specifically to a dosage form of combination of high dose high solubility active ingredient, as modified release and low dose active ingredient as immediate release suitable for swallowing; comprising of dual retard technique to control the release of high dose, high solubility active ingredient, wherein said dosage form comprising of an inner portion having a low dose active ingredient as immediate release and an outer portion having a high dose, high solubility active ingredient as modified release, in which the outer portion comprises a) micro matrix particles and b) coating on micro matrix particles.

Hilton and Deasy, [J. Pharm. Sci. 82(7):737-743 (1993)] describe a controlled-release tablet of Amoxicillin trihydrate based on the enteric polymer hydroxypropylmethyl cellulose acetate succinate. This polymer suppressed the release of the drug in the presence of gastric pH but could enhance its release in the small intestine. Single dose studies with a panel of fasting subjects showed that the tablets had a relative bioavailability of only 64.4%, probably because of the poorer absorption of Amoxicillin from the distal jejunum and ileum than from the duodenum and proximal jejunum. Other pharmacokinetic parameters confirmed a lack of therapeutic advantage of these factors over an equivalent dose of conventional capsule.

Hilton and Deasy [Int. J. Pharm. 86(7):79-88 (1992)] also describe a floating tablet of Amoxicillin trihydrate. A bilayer tablet was initially formed in which the controlled-release drug layer consisted of Amoxicillin and hydroxypropyl cellulose. This layer was bonded to a gas generating layer. However, when the two layers were joined together, the composite tablet failed to float and prematurely split along the joining of the two layers. Consequently, it was decided to abandon this approach in favor of a single-layer floating tablet. This tablet remained buoyant for 6 hours and had satisfactory in vitro sustained release. However, compared with conventional capsules in fasting humans at 500 mg equivalent dose of Amoxicillin, the relative bioavailability of the tablets were 80.5% and other pharmacokinetic parameters T (0.1 mug/ml) and T (0.5 mug/ml) corresponding to the length of time for which the serum levels remained greater than or equal to 0.1 mug/ml and 0.5 mug/ml, respectively, indicated lack of improved efficacy.

Uchida et al. [Chem. Pharm. Bull. 37(12):3416-3419 (1989)] describe a preparation of Amoxicillin, microencapsulated in ethyl cellulose. These micro-capsules exhibited a sustained-release effect when administered to dogs. However, such effect could be foreseen, since the gastric pH of the dogs which were tested, is considerably higher than human gastric pH (pH of about 6 in beagle dogs, compared to pH of about 2 in humans). The Amoxicillin is much less soluble at pH 6 than at pH 2. One would expect to obtain a very quick release of the drug from the same microcapsules if administered to humans. Hence, such combination would not provide a controlled release of Amoxicillin.

Arancibia et al. [Int. J. Clin. Pharmacol. Ther. Toxicol. 25(2):97-100 (1987)] investigated the pharmacokinetics and bioavailability of Amoxicillin trihydrate. They refer to controlled-release tablets, the composition of which is not described. In any case, no drug was detectable after 8 hours from oral administration and therefore this formulation had no advantage over conventional formulations.

Some of the compositions discussed in the art are prepared using hydrophilic swellable polymers. These compositions require the use of excessive quantities of release controlling agents. This, combined with high dose of Amoxicillin, results in a product which is too bulky to administer orally. In addition, these products have significant food effects resulting in variable bioavailability. Another approach available in the art involves the use of bioadhesive polymers. Such products are highly variable since bioadhesiveness is a property which is significantly dependent on the gastric contents. Presence of food in the stomach reduces the bioadhesive property resulting in reduced bioavailability. A third approach discussed in the art uses enteric polymers. Since Amoxicillin is predominantly absorbed from proximal part of small intestine, enteric release of the drug results in loss of bioavailability. Hence there still exists a need for developing controlled release compositions of Amoxicillin, either alone or in combination with other antibiotic(s), devoid of limitations discussed above.

### SUMMARY OF THE INVENTION

It is an objective of the present invention to provide a non-disintegrating, non-eroding, non-bioadhesive and non-swelling oral controlled release pharmaceutical composition comprising at least one high dose water soluble active ingredient, at least one diluent, at least one binder, and a polymer system comprising of at least one release controlling polymer, wherein the composition formulated into a suitable dosage form maintains its geometric shape even after the drug has diffused from the dosage form and provides the concentrations of active ingredient above effective levels for extended periods of time, optionally with other pharmaceutically acceptable excipients.

It is an objective of the present invention to provide a non-disintegrating, non-eroding, non-bioadhesive and non-swelling oral controlled release pharmaceutical composition comprising at least one high dose water soluble active ingredient, preferably antibiotic, more preferably amoxicillin or its pharmaceutically acceptable salts, hydrates, polymorphs, esters, or derivatives thereof, most preferably amoxicillin sodium; at least one diluent; at least one binder, and a polymer system comprising of at least one release controlling polymer, wherein the composition formulated into a suitable dosage form maintains its geometric shape even after the drug has diffused from the dosage form and provides the concentrations of active ingredient above effective levels for extended periods of time, optionally with other pharmaceutically acceptable excipients.

It is also an objective of the present invention to provide controlled release composition comprising an antibiotic as an active ingredient in combination with at least one other antibiotic.

It is a further objective of the present invention to provide controlled release composition, wherein the composition provides an initial burst release of approximately 20%-40% of the active ingredient within one hour for achieving blood levels equivalent to minimum inhibitory concentration, while maintaining these levels for an extended period of time.

It is yet another objective of the present invention to provide process for the preparation of such composition which comprises of the following steps:

i) mixing of active ingredient(s), diluent(s), binder(s), and polymer(s),

ii) optionally adding one or more other pharmaceutically acceptable excipients, and

iii) formulation of the mixture into a suitable dosage form.

### DETAILED DESCRIPTION OF THE INVENTION

The present invention relates to a non-disintegrating and non-eroding, non-bioadhesive and non-swelling oral controlled release pharmaceutical composition comprising at least one high dose water soluble active ingredient, at least one diluent, at least one binder, and a polymer system comprising of at least one release controlling polymer, optionally with other pharmaceutically acceptable excipients.

The composition is formulated into a suitable dosage form which maintains its geometric shape even after the drug has diffused from the dosage form and provides the concentrations of active ingredient above effective levels for extended periods of time.

The active ingredient of the present invention may be selected from but not limited to a group comprising high dose water soluble drugs such as metformin, potassium chloride, nicotinic acid, phenformin, clindamycin, ciprofloxacin, erythromycin, quetiapine, balsalazide, sodium valproate, vancomycin, or its pharmaceutically acceptable salts or derivatives thereof.

The active ingredient of the present invention is selected from a group comprising antibiotics, such as cephalosporins and penicillins, and their pharmaceutically acceptable salts, hydrates, polymorphs, esters, or derivatives thereof. The active ingredient is preferably antibiotic, more preferably amoxicillin or its pharmaceutically acceptable salts, hydrates, polymorphs, esters, or derivatives thereof, most preferably amoxicillin sodium.

In another embodiment, the present invention relates to the controlled release formulations of Amoxicillin sodium for maintaining concentrations above effective levels, for extended periods of time. The release mechanism involves predominantly diffusion and the product is in the form of a non-disintegrating tablet. The tablet maintains its geometric shape even after the drug has diffused from the system. In addition the formulation has been found to have a unique release profile with a monolithic structure. It gives an initial burst release of approximately 20%-40% within one hour for achieving blood levels equivalent to minimum inhibitory concentration, while maintaining these levels for an extended period of time. In another embodiment of the present invention, the controlled release tablets prepared using the said composition may provide better patient compliance since they need to be administered twice daily as compared to the 500 mg dose given tid.

The invention relates to the controlled release formulations of antibiotic either alone or in combination with other antibiotic(s) for maintaining concentrations above effective levels, for extended periods of time. Preferably, the invention relates to controlled release formulation of Amoxicillin sodium. The release mechanism involves predominantly diffusion and the product is in the form of a non-disintegrating tablet. The tablet maintains its geometric shape even after the drug has diffused from the system.

Nicotinic acid, also known as ‘niacin’, has been used since long in the treatment of hyperlipidemia. This compound has long been known to exhibit the beneficial effects of reducing total cholesterol, low density lipoproteins or “LDL cholesterol”, triglycerides and apolipoprotein a (Lp(a)) in the human body, while increasing desirable high density lipoproteins or “HDL cholesterol”. However, the use of nicotinic acid tends to be limited due to its side effects such as cutaneous flushing and inconvenient dosing regimens. Most of the existing formulations of nicotinic acid are hydroxypropylmethyl cellulose (HPMC) based swellable and disintegrable type dosage forms, which provide primarily an unpredictable release of the drug during extended periods of time and erratic plasma drug concentration profiles. In an embodiment, the active ingredient of the present pharmaceutical composition is nicotinic acid, or its pharmaceutically acceptable salts or derivatives thereof.

In another embodiment, the composition of the present invention provides an initial burst release of approximately 20%-40% of the active ingredient within one hour for achieving blood levels equivalent to minimum inhibitory concentration, while maintaining these levels for an extended period of time.

In an embodiment of the present invention, the controlled release composition comprises an antibiotic as an active ingredient in combination with at least one other antibiotic. The antibiotics are selected from but not limited to the group comprising amoxicillin, ampicillin, cloxacillin, clavulanic acid, cephalosporins, and the like or pharmaceutically acceptable salts or derivatives thereof.

In the present invention, the diluent is selected from but not limited to a group comprising lactose, cellulose, microcrystalline cellulose, mannitol, dicalcium phosphate, pregelatinized starch, and the like, used either alone or in combination thereof. Preferably the diluent used is lactose.

In the present invention, the binder is selected from but not limited to a group comprising polyvinylpyrrolidone, cellulose derivatives such as hydroxypropyl-methyl cellulose, methacrylic acid polymers, acrylic acid polymers, and the like.

The polymer system of the present invention comprising of at least one release controlling polymer is selected from a group comprising polyvinylpyrrolidone/ polyvinylacetate copolymer (Kollidon® SR), methacrylic acid polymers, acrylic acid polymers, cellulose derivatives, and the like. Preferably the polymer system comprises methacrylic acid polymer, and polyvinylpyrrolidone/polyvinylacetate copolymer. More preferably, the polymer system comprises polyvinylpyrrolidone/polyvinylacetate copolymer. The methacrylic acid polymer is selected from a group comprising but not limited to Eudragit® (Degussa) such as Ammonio Methacrylate Copolymer type A USP (Eudragit® RL), Ammonio Methacrylate Copolymer type B USP (Eudragit(® RS), Eudragit® RSPO, Eudragit(® RLPO, and Eudragit® RS30D.

The ratio of methacrylic acid polymer and polyvinylpyrrolidone/ polyvinylacetate copolymer is 20:1 to 1:20 by weight of the composition, preferably 10:1 to 1:10 by weight of the composition.

The pharmaceutically acceptable excipients of the present invention are selected from the group comprising diluents, disintegrants, binders, fillers, bulking agent, anti-adherents, anti-oxidants, buffering agents, colorants, flavoring agents, coating agents, plasticizers, organic solvents, stabilizers, preservatives, lubricants, glidants, chelating agents, and the like known to the art.

In an embodiment, the lubricant(s) used in the present invention are selected from, but not limited to a group comprising of stearic acid, magnesium stearate, zinc stearate, glyceryl behenate, cetostearyl alcohol, hydrogenated vegetable oil, and the like used either alone or in combination thereof.

In an embodiment of the present invention is provided a process for preparation of a composition which comprises of the following steps:

i) mixing of active ingredient(s), diluent(s), binder(s), and polymer(s),

ii) optionally adding one or more other pharmaceutically acceptable excipients, and

iii) formulation of the mixture into a suitable dosage form.

In an embodiment, the composition of the present invention is in the form of tablets. The tablets can be prepared by either direct compression, dry compression (slugging), or by granulation. In a preferred embodiment of the present invention, the oral composition is in the form of directly compressed tablets.

The granulation technique is either aqueous or non-aqueous. Preferably, the tablets of the present invention are prepared by non-aqueous granulation technique. The non-aqueous solvent used is selected from a group comprising ethanol or isopropyl alcohol.

The present invention relates to controlled release formulation of antibiotic, either alone or in combination with other antibiotic(s), which is a non-mucoadhesive, non-disintegrating, non-swelling and non-eroding product.

In an embodiment, the invention describes controlled release non-mucoadhesive, non-disintegrating, non-swelling & non-eroding type formulation of Amoxicillin sodium. The said composition retains its geometric shape throughout its stay in the gastro-intestinal tract. The product also has the advantage of showing minimal food effect. The drug release from the product is predominantly by diffusion mechanism.

The controlled release formulations prepared according to the said invention does not loose its geometric shape throughout its transit in the gastro-intestinal tract. Such a formulation does not involve the use of swellable polymers, hydrophobic waxy materials or mucoadhesive agents. The controlled release composition of the present invention may be formulated as oral dosage forms such as tablets, capsules and the like. The examples given below serve to illustrate embodiments of the present invention. However they do not intend to limit the scope of present invention.

### EXAMPLES

### Example 1

| S. No. | Ingredient | mg/tablet |
|---|---|---|
| i) | Amoxicillin sodium(equivalent to 750 mg | 797.00 |
| | Amoxycillin) | |
| ii) | Lactose | 100.00 |
| iii) | Polyvinylpyrrolidone/Polyvinylacetate | 200.00 |
| | (PVP/PVA) co-polymer (Kollidon ® SR) | |
| iv) | Polyvinylpyrrolidone (PVP) | 50.00 |
| v) | Magnesium stearate | 10.00 |
| vi) | Talc | 10.00 |

Sift ingredients (i) to (vi). Separately blend (i), (ii), (iii) and (iv). Slug and de-slug the blend. Mix with ingredients (v) and (vi), previously sifted & kept separately. Compress into tablets.

### Example 2

| S. No. | Ingredient | mg/tablet |
|---|---|---|
| i) | Amoxicillin sodium(equivalent to 750 | 797.00 |
| | mg Amoxycillin) | |
| ii) | Lactose | 150.00 |
| iii) | Eudragit ® RS | 75.00 |
| iv) | Eudragit ® RL | 150.00 |
| v) | Polyvinylpyrrolidone (PVP) | 50.00 |
| vi) | Isopropyl alcohol | Lost in |
| | | processing |
| vii) | Magnesium stearate | 10.00 |
| viii) | Talc | 10.00 |

Sift ingredients (i), (ii), (iii) & (iv) and blend. Dissolve (v) in (vi) and granulate the blend. Dry and size the granules. Mix with ingredients (vii) and (viii) previously sifted & kept separately. Compress into tablets.

### Example 3

| S. No. | Ingredient | mg/tablet |
|---|---|---|
| i) | Amoxicillin sodium(equivalent to 500 | 530.00 |
| | mg Amoxycillin) | |
| ii) | Lactose | 50.00 |
| iii) | Polyvinylpyrrolidone/Polyvinylacetate | 125.00 |
| | (PVP/PVA) co-polymer | |
| | (Kollidon ® SR) | |
| iv) | Eudragit ® RS | 25.00 |
| v) | Polyvinylpyrrolidone | 10.00 |
| vi) | Magnesium stearate | 5.00 |
| vii) | Talc | 5.00 |

Sift ingredients (i) to (vi). Separately blend (i), (ii), (iii), (iv) and (v). Slug and de-slug the blend. Mix with ingredients (vi) and (vii), previously sifted & kept separately. Compress into tablets.

### Example 4

| S. No. | Ingredient | mg/tablet |
|---|---|---|
| i) | Amoxicillin sodium(equivalent to | 530.00 |
| | 500 mg Amoxycillin) | |
| ii) | Lactose | 100.00 |
| iii) | Eudragit ® RS | 50.00 |
| iv) | Eudragit ® RL | 100.00 |
| v) | Polyvinylpyrrolidone (PVP) | 25.00 |
| vi) | Isopropyl alcohol | Lost in |
| | | processing |
| vii) | Magnesium stearate | 5.00 |
| vii) | Talc | 5.00 |

Sift ingredients (i), (ii), (iii) & (iv) and blend. Dissolve (v) in (vi) and granulate the blend. Dry and size the granules. Mix with ingredients (vii) and (viii) previously sifted & kept separately. Compress into tablets.

### Example 5

| S. No. | Ingredient | mg/tablet |
|---|---|---|
| i) | Amoxicillin sodium(equivalent to | 530.00 |
| | 500 mg Amoxycillin) | |
| ii) | Lactose | 100.00 |
| iii) | Eudragit ® RS | 150.00 |
| iv) | Polyvinylpyrrolidone (PVP) | 25.00 |
| v) | Isopropyl alcohol | Lost in |
| | | processing |
| vi) | Magnesium stearate | 5.00 |
| vii) | Talc | 5.00 |

Sift ingredients (i), (ii) & (iii) and blend. Dissolve (iv) in (v) and granulate the blend. Dry and size the granules. Mix with ingredients (vi) and (vii), previously sifted & kept separately. Compress into tablets.

### Example 6

A Composition of Amoxicillin controlled release granules

| S. No. | Ingredient | mg/tablet |
|---|---|---|
| i) | Amoxicillin sodium (equivalent to | 530.00 |
| | 500 mg Amoxycillin) | |
| ii) | Lactose | 100.00 |
| iii) | Polyvinylpyrrolidone/Polyvinylacetate | 175.00 |
| | (PVP/PVA) co-polymer | |
| iv) | Polyvinylpyrrolidone (PVP) | 25.00 |
| vi) | Isopropyl alcohol | Lost in |
| | | processing |
| vii) | Magnesium stearate | 5.00 |
| vii) | Talc | 5.00 |
| B | Clavulanate potassium/ | 250.00 |
| | Microcrystalline cellulose 1:1 mixture | |
| | (equivalent to 125 mg Clavulanic acid) | |

### Procedure:

1. Sift ingredients A (i), A(ii) & A(iii) and blend. Dissolve A(iv) in A(v) and granulate the blend. Dry and size the granules. Mix with ingredients A (vi) and A(vii), previously sifted.
2. Sift the blend B.
3. Compress the granules of step 1 and step 2 into inlay tablets, where the Clavulanate potassium blend is inlayed into the tablet of Amoxicillin granules.

### Example 7

A Composition of Amoxicillin controlled release granules

| S. No. | Ingredient | mg/tablet |
|---|---|---|
| i) | Amoxicillin sodium (equivalent to | 530.00 |
| | 500 mg Amoxycillin) | |
| ii) | Lactose | 100.00 |
| iii) | Polyvinylpyrrolidone/Polyvinylacetate | 175.00 |
| | (PVP/PVA) co-polymer | |
| iv) | Polyvinylpyrrolidone (PVP) | 25.00 |
| vi) | Isopropyl alcohol | Lost in |
| | | processing |
| vii) | Magnesium stearate | 5.00 |
| vii) | Talc | 5.00 |

### Procedure:

1. Sift ingredients (i), (ii) & (iii) and blend.
2. Dissolve (iv) in (v) and granulate the blend.
3. Dry and size the granules and mix with ingredients (vi) and (vii), previously sifted.

B Composition of Amoxicillin controlled release granules

| S. No. | Ingredient | mg/tablet |
|---|---|---|
| i) | Clavulanate potassium/ | 250.00 |
| | Microcrystalline cellulose 1:1 mixture | |
| | (equivalent to 125 mg Clavulanic acid) | |
| ii) | Croscarmellose sodium | 50.00 |
| iii) | Talc | 10.00 |
| iv) | Magnesium stereate | 10.00 |

### Procedure:

1. Mix (i), (ii), (iii) and (iv)
2. Slug and de-slug the blend of step 1 and pass through sieve of mesh size 30.
   C. Compression into Bilayer Tablets
Compress the granules of Amoxicillin controlled release granules and Clavulanate potassium granules into bilayer tablets.

### Example 8

| Ingredients | Quantity/tablet (mg) |
|---|---|
| Nicotinic acid | 500.00 |
| Lactose | 85.00 |
| Methacrylic acid copolymer | 60.00 |
| (Eudragit ® RSPO) | |
| Stearic acid | 20.00 |
| Isopropyl alcohol (IPA) | q.s. |
| Dichloromethane | q.s. |
| Magnesium stearate | 10.00 |
| Stearic acid | 20.00 |

### Procedure:

1. Mix Nicotinic acid, Lactose and Eudragit® RSPO (40 mg) and pass through mesh size 40.
2. Dissolve Eudragit® RSPO (20 mg) and Stearic acid in IPA and Dichloromethane.
3. Granulate the material of step 1 with the material of step 2 and dry the granules.
4. After drying the granules, pass them through a sieve of mesh size 60.
5. Pass Magnesium stearate and Stearic acid through sieve of mesh size 40 and mix with the dried granules.
6. Compress blended mass into tablet.
7. Cure the tablets at 60° C. for 18 hours.

### Example 9

| Ingredients | Quantity/tablet (mg) |
|---|---|
| Ciprofloxacin | 500.00 |
| Lactose | 55.00 |
| Methacrylic acid copolymer | 30.00 |
| (Eudragit ® RSPO) | |
| Methacrylic acid copolymer | 20.00 |
| (Eudragit ® RLPO) | |
| Stearic acid | 20.00 |
| Isopropyl alcohol (IPA) | q.s. |
| Dichloromethane | q.s. |
| Magnesium stearate | 10.00 |
| Stearic acid | 25.00 |

### Procedure:

1. Mix Ciprofloxacin, Lactose and Eudragit® RSPO (20 mg) and pass through mesh size 40.
2. Dissolve Eudragit® RSPO (10 mg), Eudragit® RLPO and Stearic acid in IPA and Dichloromethane
3. Granulate the material of step 1 with the material of step 2 and dry the granules.
4. After drying the granules, pass them through a sieve of mesh size 60.
5. Pass Magnesium stearate and Stearic acid through sieve of mesh size 40 and mix with the dried granules.
6. Compress blended mass into tablet.
7. Cure the tablets at 60° C. for 18 hours.

### Example 10

| Ingredients | Quantity/tablet (mg) |
|---|---|
| Nicotinic acid | 500.00 |
| Lactose | 65.00 |
| Methacrylic acid copolymer | 40.00 |
| (Eudragit ® RSPO) | |
| Methacrylic acid copolymer | 20.00 |
| (Eudragit ® RLPO) | |
| Ethyl cellulose | 10.00 |
| Isopropyl alcohol (IPA) | q.s. |
| Magnesium stearate | 100.00 |
| Stearic acid | 20.00 |

### Procedure:

1. Mix Nicotinic acid, Lactose and Eudragit® RSPO and pass through mesh size 40.
2. Dissolve Eudragit® RLPO and Ethyl cellulose in IPA and Dichloromethane.
3. Granulate the material of step 1 with the material of step 2 and dry the granules.
4. After drying the granules, pass them through a sieve of mesh size 60.
5. Pass Magnesium stearate and Stearic acid through sieve of mesh size 40 and mix with the dried granules.
6. Compress blended mass into tablet.
7. Cure the tablets at 60° C. for 18 hours.

### Example 11

| Ingredients | Quantity/tablet (mg) |
|---|---|
| Erythromycin | 500.00 |
| Lactose | 65.00 |
| Methacrylic acid copolymer | 40.00 |
| (Eudragit ® RSPO) | |
| Methacrylic acid copolymer | 20.00 |
| (Eudragit ® RLPO) | |
| Ethyl cellulose | 10.00 |
| Isopropyl alcohol (IPA) | q.s. |
| Dichloromethane | q.s. |
| Magnesium stearate | 10.00 |
| Glyceryl behenate | 20.00 |

### Procedure:

1. Mix Erythromycin, Lactose and Eudragit® RSPO and pass through mesh size 40.
2. Dissolve Eudragit® RLPO and Ethyl cellulose in IPA and Dichloromethane.
3. Granulate the material of step 1 with the material of step 2 and dry the granules.
4. After drying the granules, pass them through a sieve of mesh size 60.
5. Pass Magnesium stearate and Glyceryl behenate through sieve of mesh size 40 and mix with the dried granules.
6. Compress blended mass into tablet.
7. Cure the tablets at 60° C. for 18 hours.

### Example 12

| Ingredients | Quantity/tablet (mg) |
|---|---|
| Nicotinic acid | 500.00 |
| Lactose | 65.00 |
| Methacrylic acid copolymer | 40.00 |
| (Eudragit ® RSPO) | |
| Methacrylic acid copolymer | 20.00 |
| (Eudragit ® RLPO) | |
| Ethyl cellulose | 10.00 |
| Isopropyl alcohol (IPA) | q.s. |
| Dichloromethane | q.s. |
| Magnesium stearate | 10.00 |
| Cetostearyl alcohol | 20.00 |

### Procedure:

1. Mix Nicotinic Acid, Lactose and Eudragit® RSPO and pass through mesh size 40.
2. Dissolve Eudragit® RLPO and Ethyl cellulose in IPA and Dichloromethane.
3. Granulate the material of step 1 with the material of step 2 and dry the granules.
4. After drying the granules, pass them through a sieve of mesh size 60.
5. Pass Magnesium stearate and Cetostearyl alcohol through sieve of mesh size 40 and mix with the dried granules.
6. Compress blended mass into tablet.
7. Cure the tablets at 60° C. for 18 hours.

### Example 13

| Ingredients | Quantity/tablet (mg) |
|---|---|
| Niacin | 500.00 |
| Lactose | 75.00 |
| Methacrylic acid copolymer | 40.00 |
| (Eudragit ® RSPO) | |
| Methacrylic acid copolymer | 30.00 |
| (Eudragit ® RLPO) | |
| Purified water | q.s. |
| Sodium hydroxide | q.s. |
| Stearic acid | 20.00 |
| Magnesium stearate | 10.00 |
| Stearic acid | 20.00 |

### Procedure:

1. Pass Niacin, Lactose, Stearic acid and Eudragitg RSPO through mesh size 40 and mix.
2. Disperse Eudragitg RS30D in water and neutralize Eudragit® RS30D with Sodium hydroxide. Granulate the bulk of step 1.
3. Dry the granules and pass through mesh size 16.
4. Pass Magnesium stearate and Stearic acid through sieve of mesh size 40 and mix with dried granules.
5. Compress blended mass into tablet.
6. Cure the tablets at 60° C. for 18 hours.

### Example 14

| Ingredients | Quantity/tablet (mg) |
|---|---|
| Metformin Hydrochloride | 500.00 |
| Lactose | 85.00 |
| Methacrylic acid copolymer | 60.00 |
| (Eudragit ® RSPO) | |
| Stearic acid | 20.00 |
| Magnesium stearate | 10.00 |
| Isopropyl alcohol (IPA) | q.s. |
| Dichloromethane | q.s. |
| Magnesium stearate | 10.00 |
| Stearic acid | 20.00 |

### Procedure:

1. Mix Metformin Hydrochloride, Lactose and Eudragit® RSPO (40 mg) and pass through mesh size 40.
2. Dissolve Eudragit® RSPO (20 mg) and Stearic acid in IPA and Dichloromethane.
3. Granulate the material of step 1 with the material of step 2 and dry the granules.
4. After drying the granules, pass them through a sieve of mesh size 60.
5. Pass Magnesium stearate and Stearic acid through sieve of mesh size 40 and mix with the dried granules.
6. Compress blended mass into tablet.
7. Cure the tablets at 60° C. for 18 hours.

### Example 15

| Ingredients | Quantity/tablet (mg) |
|---|---|
| Metformin Hydrochloride | 500.00 |
| Lactose | 65.00 |
| Methacrylic acid copolymer | 40.00 |
| (Eudragit ® RSPO) | |
| Methacrylic acid copolymer | 20.00 |
| (Eudragit ® RLPO) | |
| Ethyl cellulose | 10.00 |
| Isopropyl alcohol (IPA) | q.s. |
| Dichloromethane | q.s. |
| Magnesium stearate | 10.00 |
| Glyceryl behenate | 20.00 |

### Procedure:

1. Mix Metformin Hydrochloride, Lactose and Eudragit® RSPO and pass through mesh size 40.
2. Dissolve Eudragit® RLPO and Ethyl cellulose in IPA and Dichloromethane.
3. Granulate the material of step 1 with the material of step 2 and dry the granules.
4. After drying the granules, pass them through a sieve of mesh size 60.
5. Pass Magnesium stearate and Glyceryl behenate through sieve of mesh size 40 and mix with the dried granules.
6. Compress blended mass into tablet.
7. Cure the tablets at 60° C. for 18 hours.

1. A non-disintegrating, non-eroding, non-bioadhesive and non-swelling oral controlled release pharmaceutical composition comprising at least one high dose water soluble active ingredient, at least one diluent, at least one binder, and a polymer system comprising of at least one release controlling polymer, wherein the composition formulated into a suitable dosage form maintains its geometric shape even after the drug has diffused from the dosage form and provides the concentrations of active ingredient above effective levels for extended periods of time, optionally with other pharmaceutically acceptable excipients.

2. A composition according to claim 1, wherein said active ingredient is selected from a group comprising antibiotics, such as cephalosporins and penicillins, and their pharmaceutically acceptable salts, hydrates, polymorphs, esters, or derivatives thereof.

3. A composition according to claim 1, wherein said active ingredient is Amoxicillin sodium.

4. A composition according to claim 1, wherein said active ingredient is nicotinic acid, or its pharmaceutically acceptable salts or derivatives thereof.

5. A composition according to claim 1, wherein the composition provides an initial burst release of approximately 20%-40% of the active ingredient within one hour for achieving blood levels equivalent to minimum inhibitory concentration, while maintaining these levels for an extended period of time.

6. A composition according to claim 1, which comprises at least two active ingredients selected from the group comprising amoxicillin, ampicillin, cloxacillin, clavulanic acid, and cephalosporins, or pharmaceutically acceptable salts or derivatives thereof.

7. A composition according to claim 1, wherein the diluent is selected from a group comprising lactose, cellulose, microcrystalline cellulose, mannitol, dicalcium phosphate, pregelatinized starch, used either alone or in combination thereof.

8. A composition according to claim 1, wherein the binder is selected from a group comprising polyvinylpyrrolidone, cellulose derivatives, methacrylic acid polymers, and acrylic acid polymers.

9. A composition according to claim 1, wherein the polymer system comprises of polymers selected from a group comprising polyvinylpyrrolidone/polyvinylacetate copolymer; methacrylic acid polymers, acrylic acid polymers, and cellulose derivatives, or mixtures thereof.

10. A composition according to claim 9, wherein the polymer system comprises polyvinylpyrrolidone/polyvinylacetate copolymer.

11. A composition according to claim 9, wherein the polymer system comprises methacrylic acid polymer and polyvinylpyrrolidone/polyvinyl acetate copolymer.

12. A composition according to claim 11, wherein the methacrylic acid polymer is selected from a group comprising Ammonio Methacrylate Copolymer type A USP and Ammonio Methacrylate Copolymer type B USP.

13. A composition according to claim 1, wherein the pharmaceutically acceptable excipients are selected from the group comprising disintegrants, binders, fillers, bulking agent, coating agents, plasticizers, organic solvents, colorants, stabilizers, preservatives, lubricants, glidants, and chelating agents.

14. A composition as in claim 1, which is formulated as tablets, capsules and the like.

15. A composition according to claim 14, which is in the form of directly compressed tablets.

16. A process for preparation of a composition according to claim 1, which comprises of the following steps:
i) mixing of active ingredient(s), diluent(s), binder(s) and polymer(s),
ii) optionally adding one or more other pharmaceutically acceptable excipients, and
iii) formulation of the mixture into a suitable dosage form.

17. A method of treatment of bacterial infections and for eradication of helicobacter pylori in peptic ulcer disease by administering to a patient in need thereof a pharmaceutical composition according to claim 1.

18. (canceled)

19. A composition according to claim 6, which is formulated as tablets, capsules and the like.

20. A composition according to claim 20, which is in the form of directly compressed tablets.
